(19) United States
(12) Patent Application Publication
(10) Pub. No.: US 2017/0173274 A1
(43) Pub. Date: Jun. 22, 2017

(54) PORTABLE, PERSONAL, REUSABLE, CRYOTHERAPY UNIT INCLUDING A PRE-INJECTION SITE DERMAL NUMBING AND MARKING UNIT AND METHOD OF USING THE SAME

(71) Applicant: Ice Shot, LLC., Oakmont, PA (US)

(72) Inventors: John R. McCollum, Oakmont, PA (US); Ryan Bookhamer, Edinboro, PA (US); Joseph Chabal, Gibsonia, PA (US)

(21) Appl. No.: 15/367,875

(22) Filed: Dec. 2, 2016

Related U.S. Application Data
(60) Provisional application No. 62/262,489, filed on Dec. 3, 2015.

(51) Int. Cl. A61M 5/42

(52) U.S. Cl. CPC A61M 5/422; A61M 2205/36

(57) ABSTRACT

A portable, personal, reusable, cryotherapy unit in the form of a pre-injection site dermal numbing and marking unit includes an insulating sealable freezable rigid container, generally a plastic cylindrical structure, a freezable fluid or gel within the rigid container, and a high heat transferring applicator end having a curved application surface. A method pre-injection site treatment and periodic post injection site treatment is disclosed.

### RELATED APPLICATIONS

This application claims priority to U.S. Patent Application Ser. No. 62/262,489 filed Dec. 3, 2015, entitled “Portable, Personal, Reusable, Pre-Injection Site Dermal Numbing and Marking Unit and Method of Using the Same” which application is incorporated herein by reference in its entirety.

### BACKGROUND INFORMATION

1. Field of the Invention

The present invention relates to cryotherapy units such as for hypodermic needle injection site treatment and, in particular, to a portable, personal, reusable, pre-injection site dermal numbing and marking unit and method of using the same.

2. Background Information

Cryotherapy is the local or general use of low temperatures in medical therapy. The use of cold to relieve pain has been employed since at least Hippocrates in the 4th century BC. Often a cryotherapy device, often an ice pack, is placed over an injured area and is intended to absorb heat of a closed traumatic or edematous injury by using conduction to transfer thermal energy. The physiologic effects of cold application include immediate vasoconstriction with reflexive vasodilation, decreased local metabolism and enzymatic activity, and decreased oxygen demand. Cold decreases muscle spindle fiber activity and slows nerve conduction velocity, therefore it is often used to decrease spasticity and muscle guarding. It is commonly used to alleviate the pain of minor injuries, as well as decrease muscle soreness. One area of cryotherapy is pre-injection hypodermic needle injection site treatment.

All hypodermic needle injections may cause some discomfort to the patient as well as post injection ecchymosis and swelling. Topical anesthetics and ice are the most often employed methods for limiting the degree of pain, ecchymosis, or swelling. Much research has been devoted to the development of effective topical anesthetics of the skin to minimize or eliminate the discomfort experienced by the patient during hypodermic needle injection and similar procedures. Effective anesthesia via topical administration is difficult to achieve on keratinized or non-traumatized skin due to limited trans-epidermal absorption. Various creams, ointments, and gels have been used for this; however, their efficacy has been less than ideal and complications have occurred. While a variety of topical anesthetics are currently available, their use is limited by variable efficacy, lengthy application times, and often elaborate and time-consuming techniques for occlusion and removal of the anesthetic.

Additionally complicating the issue of pain management of hypodermic needle injection site is the increase of self-administered injections. A large number of drugs, such as insulin treatments, are self-administered via injection and the patients will typically lack medical training, nor will they have access to expensive medical equipment. Consequently, there has been an increased need for safe, effective, expedient and easily (laymen) applicable topical anesthesia.

As noted above, dermal cooling has long been proposed for pre-injection site treatment. For example, the American Society for Dermatologic Surgery (ASDS) Guidelines recommends several mechanisms to diminish dermal filler injection pain including pre-injection application of ice. For a discussion of the benefits of pre-injection dermal tissue cooling, see the paper by Mark S. Nestor, MD, PhD, Glynis R. Ablon, MD, and Mark A. Stillman, PhD entitled The Use of a Contact Cooling Device to Reduce Pain and Ecchymosis Associated With Derma/Filler Injections, J Clin Aesthet Dermatol. 2010 March, 3(3): 29-34. For additional medical studies in this area see Goel S, Chang B, Bahn K, El-Hindi N, Koli S. “Cryoanalgesic preparation” before local anesthetic injection for lid surgery, Orbit, 2006, 25:107-110; Elibol O, Ozkan B, Hekimhan P K, Caglar Y. Efficacy of skin cooling and EMLA cream application for pain relief of periocular botulinum toxin injection, Ophthalmic Plast Reconstr Surg. 2007, 23:130-133; Seo D W, Hong J P The use of topical skin cooling device to achieve pain relief of injection induced pain, Plas Reconstruc Surg. 2009, 123:111e-112e; and Fisher G H, Kim K H, Bernstein L J, Geronemus R G. Concurrent use of a handheld forced cold air device minimizes patient discomfort during fractional photothermolysis, Dermatologic Surg. 2005, 31:1242-1244.

The medical efficacy of cooling dermal tissue as pre-injection site treatment is well established, yet there are few devices to easily achieve this result. One of the above studies, The Use of a Contact Cooling Device to Reduce Pain and Ecchymosis Associated With Derma/Filler Injections, noted that, “unfortunately, the effect of applying ice or cooled air is often unpredictable because these modalities cannot be delivered accurately and precisely to targeted areas.” Further, ice itself is difficult to handle and the melting is messy and problematic.

The ArTek Spot® cryotherapy unit, from ThermoTek, Inc. of Flower Mound, Tex., is a commercially available spot contact cooling system, and has been used to control pain associated with some hair treatments and tattoo removal procedures. It also has been used to prevent pain and discomfort associated with dermal fillers and other injectable medicaments, as an alternative to ice cubes, topical ointments, and chemical sprays. Although reasonably effective in commercial or clinical settings, this 15 lbs unit (with a 7 foot hose) is bulky and simply not applicable for most consumer or self-administered injections. The size and cost has limited the wide acceptance or adoption of this device in some clinical settings as well.

Thus there remains a need for an inexpensive, efficient and effective spot cooling method and apparatus for pain management or other cryotherapy applications usable by laymen and which minimizes or eliminates the mess of melting ice cubes or chemical sprays.

### SUMMARY OF THE INVENTION

The present invention provides a portable, personal, reusable, cryotherapy unit such as a pre-injection site dermal numbing and marking unit and method of using the same. Unless otherwise noted, “injection” and “injection site” will reference hypodermic needle injections and hypodermic needle injection sites.

One aspect of the present invention provides a portable, personal, reusable, pre-injection site dermal numbing and marking unit includes an insulating sealable freezable rigid container, a freezable fluid or gel within the rigid container and a high heat transferring applicator end having a curved application surface.

One aspect of the invention is described as providing A portable, personal, reusable, pre-injection site dermal numbing and marking unit comprising: an insulating sealable freezable rigid plastic container; a transferring applicator end having a curved application surface and a gel housing, wherein the gel housing is partially contained within the rigid plastic container; a freezable gel within the gel housing, wherein the gel housing does not extend the full length of the rigid housing and wherein the rigid plastic housing is configured to be gripped in locations laterally spaced from the gel housing.

One aspect of the invention provides a method injection site treatment includes the steps of: Providing a portable, personal, reusable, pre-injection site dermal numbing and marking unit includes an insulating sealable freezable container, a freezable fluid or gel within the container and a high heat transferring applicator end having a curved application surface; Placing the dermal numbing and marking unit within a freezing appliance; Removing the dermal numbing and marking unit from the freezing appliance and exposing the application surface; Holding the application surface against dermas at the injection site for a fixed period prior to injection; and Removing the dermal numbing and marking unit from the injection site.

The method of injection site treatment according to invention may further include post injection site treatment comprising holding the application surface against dermas at the injection site for a fixed period following an injection.

These and other aspects of the present invention will be clarified in the description of the preferred embodiment of the present invention described below in connection with the attached figures in which like reference numerals represent like elements throughout.

### BRIEF DESCRIPTION OF THE DRAWINGS

FIG. 1A is a perspective view of a portable, personal, reusable, cryotherapy unit in the form of a pre-injection site dermal numbing and marking unit without a cap according to one embodiment of the present invention;

FIG. 1B is a side elevation view the dermal numbing and marking unit, without a cap, of FIG. 1A;

FIG. 1C is a top plan view the dermal numbing and marking unit, without a cap, of FIG. 1A;

FIG. 1D is a bottom plan view the dermal numbing and marking unit of FIG. 1A;

FIG. 2A is a perspective view of a portable, personal, reusable, cryotherapy unit in the form of a pre-injection site dermal numbing and marking unit according to one embodiment of the present invention;

FIG. 2B is a side elevation view the dermal numbing and marking unit of FIG. 2A with the cap removed;

FIG. 2C is a side elevation sectional view of the dermal numbing and marking unit of FIG. 2A without the cap;

FIG. 3 is a side elevation sectional view of a portable, personal, reusable, cryotherapy unit in the form of a pre-injection site dermal numbing and marking unit, without a cap, according to one embodiment of the present invention;

FIG. 4 is a side elevation, partially exploded, view of a portable, personal, reusable, cryotherapy unit in the form of a pre-injection site dermal numbing and marking unit according to one embodiment of the present invention;

FIG. 5 is a perspective schematic view of a portable, personal, reusable, cryotherapy unit which may form a pre-injection site dermal numbing and marking unit, with the cap omitted for clarity, according to another embodiment of the present invention;

FIG. 6A is a side elevation sectional view of an unfilled, refillable, portable, personal, reusable, cryotherapy unit in the form of a pre-injection site dermal numbing and marking unit, without a cap, according to one embodiment of the present invention;

FIG. 6B is a side elevation sectional view of a modified version of the pre-injection site dermal numbing and marking unit of FIG. 6A, with a molding cap, according to one embodiment of the present invention;

FIG. 7 is a side elevation sectional view of a portable, personal, reusable, cryotherapy unit in the form of a pre-injection site dermal numbing and marking unit, without a cap, according to one embodiment of the present invention;

FIG. 8A is a side elevation of a portable, personal, reusable, cryotherapy unit in the form of a pre-injection site dermal numbing and marking unit, without a cap, according to one embodiment of the present invention;

FIG. 8B is a top plan view the dermal numbing and marking unit, without a cap, of FIG. 8A;

FIG. 9A is a side elevation of a portable, personal, reusable, cryotherapy unit, without a cap, according to one embodiment of the present invention;

FIG. 9B is a top plan view the cryotherapy unit, without a cap, of FIG. 9A;

FIG. 9C is a perspective partially section view of the cryotherapy unit, without a cap, of FIG. 9A; and

FIG. 10 is a perspective view of the shipping package of the dermal numbing and marking units of FIG. 2A.

### BRIEF DESCRIPTION OF THE PREFERRED EMBODIMENTS

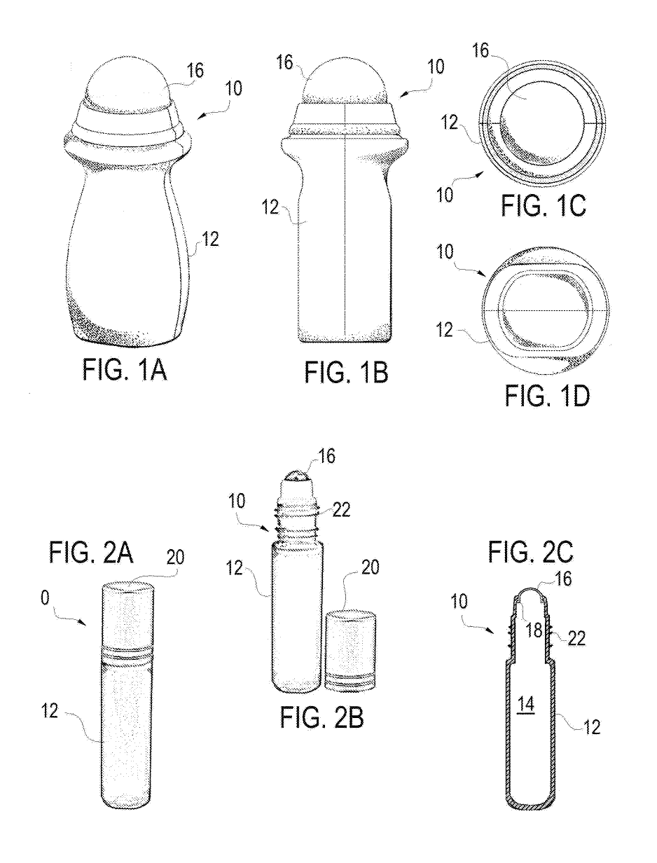

As shown in the distinct embodiments of FIGS. 1A-D, 2A-C, 3, 4, 5, 6A, 6B, 7, 8A-B, and 9A-C this invention is a portable, personal, reusable, cryotherapy device generally in the form of a pre-injection site dermal numbing and marking unit 10 which includes an insulating sealable freezable container 12, a freezable fluid or gel 14 within the container 12, and a high heat transferring applicator end 16 having a curved application surface.

The container 12 is a rigid structure and may be formed of any material, but injection molded plastic is likely the most cost effective and suitable for the intended use of unit 10. The term rigid means it will hold its shape and the container 12 is sufficient to form a handle for the unit 10. A generally cylindrical shape, as shown in the embodiments of FIGS. 1A-D, 2A-C, 3, 4, 6A, 6B, 7, 8A-B, and 9A-C, is deemed convenient and easy to grip and hold, however an oblong oval shape in cross section for the container 12 can match a larger applicator end 16, as shown in the embodiment of FIG. 5. Regarding the size of the cylindrical embodiments of the rigid container 12, any desired size is possibly, but generally ¾″-2″ will be easily gripped and provide for a sufficient amount of fluid or gel 14 in the interior with appropriate walls. The base and walls of the cylinder of container 12 to the threads 22 may be a generally thicker wall construction to allow for greater insulating capabilities for the fluid or gel 14. The length of the container 12 can be any convenient length for carrying and application, but 2″-6″ yields a useful range in heights for container 12.

The freezable fluid or gel 14 within the rigid container 12 (either directly or within a separate housing 19 as discussed below) may be water, or preferably is a freezable gel such as silica gel, hydroxyethyl cellulose, Sodium polyacrylate or other polymer. Preservatives may be used within the fluid or gel 14 to prevent bacteria growth, particularly in the manufacturer sealed embodiments (FIGS. 1A-D, 2A-C, 3, 4, 5, 7, 8A-B, and 9A-C) for the container 12 of the present invention. The fluid or gel 14 materials, once cooled, can absorb a considerable amount of heat before they warm above 0 C, due to the high latent heat of fusion. Suitable gel materials are those used in gel filled cold packs. The freezable fluid or gel 14 in the embodiment of FIGS. 1A-D, 2A-C, 3, 4, 5, 7, 8A-B, and 9A-C are sealed within the container 12 by the manufacturer. As discussed below a user fillable version, such as shown in the embodiments of FIGS. 6A and 6B allows the user to fill the container 12 with the fluid or gel 14, which is generally water and aqueous mixtures for these user filled embodiments. Any desired fluid or gel 14 material may be used for the user supplied fluid 14 (as with the manufacturer supplied fluid or gel 14), for example a mixture of alcohol and water may easily form the fluid or gel 14 in the user filled embodiments as this combination of ingredients is often identified when describing how to easily make a homemade cold pack.

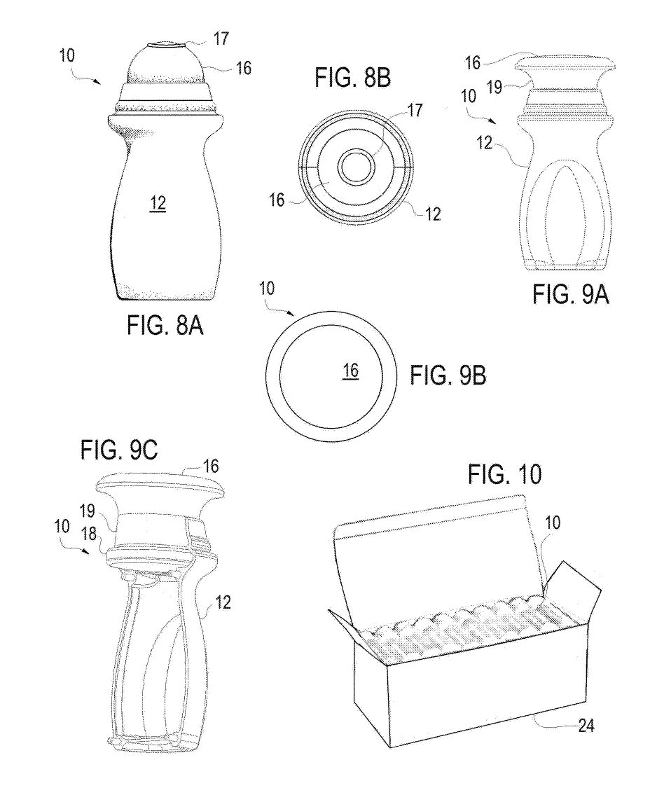

The high heat transferring applicator end 16 includes a curved application surface. The applicator end 16 may be a semi-spherical, or hemispherical, surface as shown in FIGS. 1A, 1B, 2B, 2C, 4, 6A, and 7. The applicator end 16 may be an elongated curved end as shown in FIG. 8, such as a football profile. A large radius curve is used for an enlarged applicator end 16 shown in FIGS. 9A-C. The elongated or larger shapes such as shown in FIGS. 5 and 9A-C are useful when the cryotherapy unit 10 is used for more general cryotherapy applications (e.g., on a sore muscle or joint) other than, or in addition to, pre-injection site treatment. The curved application surface of the applicator end 16 is a better applicator than a flat surface. The applicator end 16, at least the portion contacting the skin, may be made of metal, such as stainless steel, with high thermal conductivity to yield the high heat transfer property desired. The applicator end 16 need not be rigid, like the container 12, but could be flexible such as a thin membrane of rubber or plastic.

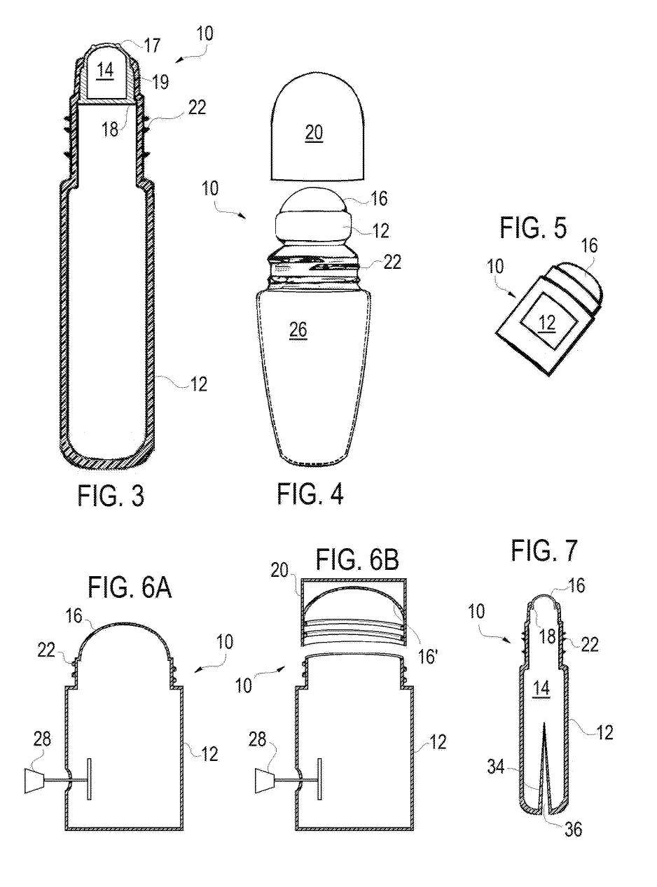

The embodiments of FIGS. 3 and 8A and B further show the applicator end 16 may include a raised ring 17 on a top surface thereof. The raised ring 17 accentuates the marking aspect of the unit 10. The application of the cold to the injection site through the use of the unit 10 will both cool and mark the skin at the injection site. The marking is typically a visible discoloration of the skin due to the cooling treatment. The shape of the mark is in the shape of the applicator end 16. The marking of the skin is helpful as it gives the user a visible indication of the location of the numbed or treated tissue, providing a visible target for administering the injection and helping to assure the user does not miss the treated tissue, which would obviate the effectiveness of the pre-injection treatment. The raised ring 17 provides a further visible circle in the treated skin to highlight the treated area and further focus the user to the proper placement of the injection site. Precision of an injection may be critical when injecting dermal fillers or Botox, for example, and the unit 10 with the ring can be used by medical professionals to increase accuracy of treatment as well as manage pain.

The applicator end 16 may be formed with a mounting flange 18 to assist the holding of the end 16 within the rigid container 12. In other embodiments, such as shown in FIGS. 3 and 9C the applicator end 16 may include an abbreviated rigid gel housing 19 containing the gel 14, with the housing 19 including a retaining flange 18 to help secure the end 16, with housing 19, in the rigid container 12. The housing 19 need not extend the length of the rigid container 12, as shown, and this allows the container 12 to further insulate the user's hands that hold the container 12 from the cooling effects of gel 14.

Alternatively the applicator end 16 may be formed of plastic such that the container 12 and applicator end 16 are integral as shown in the embodiment of FIG. 6A. In this alternative the “high heat transferring” property of the applicator end 16 of the invention is through forming the applicator 16 as a thin element relative to the body of the rigid container 12. The “high heat transferring” property is thus a relative property measured relative to the body of the rigid container 12. Whether in plastic or metal a “thin walled” for applicator end 16 is preferred to facilitate heat transfer. The high heat transferring property can also be categorized as a low insulating property or a high thermal conductive property.

For the spherical applicator ends 16, these can be of any size desired generally associated with the diameter of the container 12, however a diameter range of ⅜″-1½″ yields an effective treatment surface with a viable target marked treated area for the user.

As shown in select FIGS. 2A, B, 4, and 6B the dermal numbing and marking unit 10 includes a cap 20 that can be coupled to the container 12 such as through molded threads 22. Other coupling techniques for attaching the cap 20 to the container 12, such as a snap fit, may be implemented as known in the art.

As noted above FIG. 5 illustrates a unit 10 with a slightly different shape of container 12 namely a more oblong shape, similar to a conventional stick deodorant container. This allows for a larger oblong applicator end 16, in cross section it may be an oval of about 1¼ by 2¼″ or similar proportions, to provide a larger oblong treated area for the user. A treatment area having a major/minor axis (longer in one direction) may be preferable to some users or patients, particularly for general cryotherapy applications other than pre-injection site treatment. Similarly the embodiment of FIGS. 9A-C show an enlarged treatment applicator end 16 flaring out to a size larger than the diameter of the rigid container 12 and that will be well suited for generally cryotherapy applications.

FIG. 4 illustrates a unit 10 with a slightly different shape of container 12. The alternative shape for container 12 in this embodiment is mainly ornamental, but may yield improved handling for users. FIG. 4 further illustrates a surrounding insulating/gripping sleeve 26 surrounding the container 12. The gripping sleeve 26 will increase the insulation around the fluid 14 within the container 12 and provide a more comfortable gripping surface for the user. The gripping sleeve 26 can further provide a decorative or ornamental surface for the unit 10. The need for additional insulation on the handle portion of the container 12 is reduced when the applicator end 16 includes the gel housing 19 containing the gel 14 that does not extend the entire length of the container 12 as shown.

FIG. 6A illustrates a unit 10 which is not “factory sealed” and it includes a plug 28 to allow users to selectively fill the unit 10 with fluid 14, likely an aqueous alcohol mixture. The term sealed or sealable, as used herein, means that the fluid or gel 14 does not come into contact with the user and the applicator end 16 is a physical barrier that can be considered as part of the container structure. The embodiment of FIG. 6A is sealable (i.e. sealed and resealed) by the user via plug 28 while the embodiments of FIGS. 1A-D, 2A-C, 3, 4, 5, 7, 8A-B, and 9A-C are sealable at time of manufacture (i.e. permanently sealed).

FIG. 6B illustrates a further embodiment, which is user fillable as with FIG. 6A, but which is distinctly different from all the other embodiments in that it does not provide a sealed unit 10, and may be categorized as an “open” unit 10. In this embodiment the physical applicator end 16 as a separate element is omitted and the cap 20 includes an applicator molding surface 16′ which serves as a mold, like an ice cube tray, for the fluid 14 within the container 12. The user fills the container 12 (with cap 20 in place) of this embodiment with water or alcohol/water mixture, or any desired fluid 14 via the opening and plug 28. The user then replaces the plug 28 and places the filled unit 10 in the freezing appliance with the cap 20 side down. The fluid will freeze within the cap 20 and the container 12. In this embodiment a fluid which completely freezes must be utilized for fluid 14. The “applicator end” 16 of this embodiment is the top surface of the frozen fluid 14 formed to the shape of the mold surface 16′ and exhibits a high temperature transfer because of the direct contact between the frozen fluid 14 and the dermal tissue. In a sense, in the remaining embodiments other than that of FIG. 6B the applicator end 16 forms an extension of the container 12 and can be considered part of the container 12, while in the embodiment of FIG. 6B the applicator end 16 is integrated with the fluid 14. The applicator end 16 in the embodiment of FIG. 6B is defined by the shape of the mold surface 16′ and should follow the guidelines for the shape and size discussed above for applicator end 16. A spherical mold and applicator end 16 is shown in FIG. 6B. The unit 10 of this embodiment is used similar to the units 10 described above except that with the cap 20 removed at time of treatment the frozen fluid 14 will be in direct contact with the dermal tissue. This unit 10 has some advantages in cooling transmission through the elimination of the physical applicator end 16 as part of the container 12, but introduces the melting fluid 14 and the direct dermal contact that can be considered “messy” by some users and thus problematic as described above.

FIG. 7 is a side sectional view of a dermal numbing and marking unit 10 according to another embodiment of the present invention, wherein the cap 20 is omitted for clarity. In this unit 10 the container 12 includes a thin walled conical member 34 extending from the side opposed from the applicator end 16 forming a needle receiving aperture 36 extending into the interior of the container 12 as shown. The aperture 36 is sized to receive a conventional hypodermic needle therein. This allows the user to selectively cool an injection needle prior to injection. There is less research on the efficacy of cooling a needle prior to injection as opposed to the dermal injection site, but FIG. 7 illustrates a unit that allows the unit 10 to accommodate both dermal cooling treatments and needle cooling treatments as desired. The thin walled conical member 34 allows for relatively high heat transfer for efficient cooling of the needle, when such needle cooling is desired by the user. The unit 10 of FIG. 7 as described yields a portable, personal, reusable, pre-injection site dermal numbing and marking unit 10 with integral sealed needle cooling system. The cap 20 is omitted from the FIG. 7 and an additional bottom cap (not shown) may be utilized to cover the needle receiving aperture 36 which may assist in maintaining the unit 10 cooler for longer periods between uses. Further, it is possible to separate the needle cooling aspects of the unit 10 into its own separate device, if desired. In operation for needle cooling the cover of the hypodermic needle is removed and the needle inserted into the aperture 36 and left there for a period of time to cool the needle. As this is treating the needle and not the dermal tissue directly, there are no significant issues for having an extended treatment period, for example periods of 30 seconds to a minute or much more.

FIG. 10 is a perspective view of the shipping package of the dermal numbing and marking units of FIG. 2A further showing the compact portability of the units 10.

In operation the dermal numbing and marking unit 10 is used in a method of injection site treatment which includes placing the dermal numbing and marking unit within a freezing appliance, such as the freezing compartment of a conventional refrigerator, to freeze the gel or fluid 14. The user may then remove the dermal numbing and marking unit 10 from the freezing appliance after sufficient time for the unit 10 to reach effective temperatures (typically a few hours), and carry the unit with them (to work or along for other daily activity).

The container 12 and fluid 14 will maintain the unit 10 at an operable temperature for several hours (generally 4-6 hours for a unit shown in FIGS. 1A-D, 2A-C, 3, 4, 5, 7, 8A-B, and 9A-C and with gel material for fluid 14). The cap 20 will also have an insulating effect while the cap 20 remains in place on the container 12. The interior of the cap may be thicker or may have an insulating member such as foam added thereto to facilitate the insulation function.

Immediately prior to administering an injection the user removes the cap 20 from the unit 10 to expose the application surface on applicator end 16. The user then holds the application surface of applicator end 16 against dermal tissue at the injection site for a fixed period, typically 4-20, generally 5-10 seconds, immediately prior to hypodermic needle injection. Following the pre-injection treatment of the dermal tissue the user removes the dermal numbing and marking unit 10 from the injection site.

The method may be repeated by returning the cap 20 onto container 12 and returning the unit 20 to the freezing appliance and repeating the process.

The cryotherapy unit 10 is not limited to pre-injection treatment. Post injection pain and inflammation may also be treated with the unit 10. The method of injection site treatment according to the invention further includes post injection site treatment, as needed, comprising holding the application surface of the applicator end 16 against dermal tissue at the injection site for a fixed period following an injection.

The unit 10 is designed for use by “consumers” for self-administered injections, such as hypodermic needle injections administered by those without significant medical training. The unit 10 is not limited to use by non-medical professionals. Consider that the number of aesthetic procedures requiring the use of needle-based injections is progressively increasing. Botulinum toxins, commonly called Botox, and injectable hyaluronic acid dermal fillers represent a substantial portion of these injections. According to the American Society for Aesthetic Plastic Surgery, hyaluronic acid gels account for approximately 80 percent of the nearly two million soft tissue filler injections performed in 2006. As noted above, all injections may cause some discomfort to the patient as well as post-injection ecchymosis and swelling. The unit 10 yields an effective tool for clinicians for administering these injections. The marking can assist the clinician, or particularly an assisting technician, in proper placement of the injection. Further the unit 10 may be provided to the patient for self-administered post injection treatment, as needed.

The unit 10 as shown and described above yields a portable, personal, reusable, cryotherapy unit in the form of pre-injection site dermal numbing and marking unit 10, which can be used for periodic post injection treatments. It is apparent that many variations to the present invention may be made without departing from the spirit and scope of the invention. The present invention is defined by the appended claims and equivalents thereto.

What is claimed is:

1. A portable, personal, reusable, pre-injection site dermal numbing and marking unit includes an insulating sealable freezable rigid container, a freezable fluid or gel within the rigid container and a high heat transferring applicator end having a curved application surface.

2. The dermal numbing and marking unit according to claim 1 wherein a freezable gel is provided within the rigid container.

3. The dermal numbing and marking unit according to claim 2 wherein the high heat transferring applicator end comprises a semi-spherical skin engaging structure.

4. The dermal numbing and marking unit according to claim 3 wherein the semi-spherical skin engaging structure of the high heat transferring applicator end has a diameter in the range of ⅜″-1½″.

5. The dermal numbing and marking unit according to claim 4 wherein the semi-spherical high heat transferring applicator end is formed of one of plastic, rubber or metal.

6. The dermal numbing and marking unit according to claim 5 wherein the rigid container is generally cylindrical having a diameter of ¾″-2″ and formed of plastic.

7. The dermal numbing and marking unit according to claim 5 wherein the high heat transferring applicator end further includes a gel housing containing the gel within the rigid container.

8. The dermal numbing and marking unit according to claim 7 wherein the gel housing does not extend the full length of the rigid housing and wherein the rigid housing is configured to be gripped in locations laterally spaced from the gel housing.

9. The dermal numbing and marking unit according to claim 2 wherein the high heat transferring applicator end further includes a gel housing containing the gel within the rigid container.

10. The dermal numbing and marking unit according to claim 9 wherein the gel housing does not extend the full length of the rigid housing and wherein the rigid housing is configured to be gripped in locations laterally spaced from the gel housing.

11. The dermal numbing and marking unit according to claim 2 wherein the applicator end includes a retaining flange to help secure the end in the rigid container.

12. The dermal numbing and marking unit according to claim 2 wherein the high heat transferring applicator end comprises an oblong skin engaging structure formed in cross section as an oval of about 1¼ by 2¼″.

13. The dermal numbing and marking unit according to claim 2 wherein the high heat transferring applicator end comprises a ring of material on a skin engaging surface.

14. The dermal numbing and marking unit according to claim 1 further including a replaceable plug to allow users to fill the container with the fluid or gel prior to freezing.

15. A portable, personal, reusable, pre-injection site dermal numbing and marking unit comprising
an insulating sealable freezable rigid plastic container;
a heat transferring applicator end having a curved application surface and a gel housing, wherein the gel housing is partially contained within the rigid plastic container;
a freezable gel within the gel housing, wherein the gel housing does not extend the full length of the rigid housing and wherein the rigid plastic housing is configured to be gripped in locations laterally spaced from the gel housing.

16. The dermal numbing and marking unit according to claim 15 wherein the rigid container is generally cylindrical having a diameter of ¾″-2″.

17. The dermal numbing and marking unit according to claim 15 wherein the heat transferring applicator end comprises a semi-spherical skin engaging structure having a diameter in the range of ⅜″-1½″.

18. The dermal numbing and marking unit according to claim 15 wherein the heat transferring applicator end comprises a ring of material on a skin engaging surface.

19. A method injection site treatment includes the steps of:
Providing a portable, personal, reusable, pre-injection site dermal numbing and marking unit includes an insulating sealable freezable container, a freezable fluid or gel within the container and a high heat transferring applicator end having a curved application surface;
Placing the dermal numbing and marking unit within a freezing appliance;
Removing the dermal numbing and marking unit from the freezing appliance and exposing the application surface;
Holding the application surface against dermas at the injection site for a fixed period prior to injection; and
Removing the dermal numbing and marking unit from the injection site.

20. The method of injection site treatment according to claim 19 further including post injection site treatment comprising holding the application surface against dermas at the injection site for a fixed period following an injection.
